(19) United States
(12) Patent Application Publication
(10) Pub. No.: US 2004/0096569 A1
(43) Pub. Date: May 20, 2004

(54) Edible film products and methods of making same

(76) Inventors: David G. Barkalow, Deerfield, IL (US); Daniel J. Zyck, North Riverside, IL (US); Gary S. Kehoe, Glendale, AZ (US); Ronald Grey, Morton Grove, IL (US); Albert H. Chapdelaine, Naperville, IL (US); Donald A. Seielstad, Frankfort, IL (US); James R. Maxwell, Chicago, IL (US); Julius Zuehlke, Chicago, IL (US); Joseph D. Rancich, Park Ridge, IL (US); Scott Marske, LaGrange, IL (US); Sonya S. Johnson, LaGrange Highlands, IL (US); David J. Cai, Darien, IL (US); Gordon N. McGrew, Evanston, IL (US); Rebecca A. Aumann, Chicago, IL (US)

Correspondence Address: BELL, BOYD & LLOYD LLC, P. O. BOX 1135, CHICAGO, IL, 60690-1135, US

(21) Appl. No.: 10/295,277

(22) Filed: Nov. 15, 2002

(51) Int. Cl. A61K 47/00; A23G 3/00

(52) U.S. Cl. 426/660

(57) ABSTRACT

Edible thin films and methods of making same are provided. The edible thin films provide a variety of different products that can vary in flavor, color, shape, and mouthfeel.

### BACKGROUND OF THE INVENTION

The present invention generally relates to edible compositions. More specifically, the present invention relates to edible films and methods of making and using same.

Edible film products are known in the art. These products are designed to adhere to and rapidly dissolve in the mouth of the consumer. Edible films can provide flavor and/or oral care agents, e.g., breath freshening to the consumer. Such films typically include a film former and flavor or other ingredient. See, for example, U.S. Pat. No. 5,948,430 and U.S. Application Publication No. U.S. Pat. No. 2001/0022964 A1.

Edible film products are provided to the consumer in strip form. The strips are usually sized so that they can be placed on the tongue of a consumer. In this regard, the edible film strips typically have a size of a postage stamp or slightly larger. These strips preferably have a supple texture and are non-self adhering.

One type of edible film product is distributed by Pfizer Consumer Healthcare under the name Listerine® PocketPaks™. The Pfizer edible film product is packaged in a plastic container that includes a top that can open along a hinge. A stack of strips are located in an interior of the package one on top of another. The package is designed so that the consumer can open the container and remove one strip from the stack with his or her finger.

Although current edible film strips and packaging provide a viable product, there are some issues with the design. One issue is manufacturing the edible film strip products. A further potential issue is the way the edible film products are dispensed or accessed. As noted above, these products can be provided in a stacked formation requiring the consumer to slide off a strip from the stack with his finger. Sharing of the product by consumers can cause concerns.

Of course, it has been known to provide edible products in a variety of shapes and forms. Confectionery products have been molded, extruded, or otherwise shaped into various forms over the years. For example, bubble gum has been formed into shapes such as flat sticks, cylinders, cubes, cigars, shredded chew, and the like. By way of example, U.S. Pat. No. 4,882,175 discloses a method and an apparatus for forming a confectionery product into a rolled or tape form.

It is generally known that providing a confectionery product in a novelty form can enhance the marketability of a product, particularly to young consumers. Edible films could benefit from new, novel forms.

There is therefore a need for improved edible film products and methods of making same.

### SUMMARY OF THE INVENTION

Generally, the present invention provides improved edible film formulations and methods of making the same. Pursuant to the present invention, novel forms of consumable edible thin films are provided that can increase the marketability of the product, particularly to younger consumers, and provide, for at least certain uses, an improvement product. To this end, a variety of edible improved thin films are provided.

In an embodiment, the present invention provides an edible thin film product comprising a container including a body that defines an interior for housing the edible thin film. The edible thin film has a characteristic indicative of a flavor of the edible thin film, the characteristic being chosen from the group consisting of color and shape. The body of the container includes the characteristic.

In an embodiment, the body of the container has substantially the same cross-sectional shape as the edible thin film.

In an embodiment, the body of the container has portions thereof that are of a same color as the edible thin film.

In an embodiment, the edible thin film provides at least two flavors.

In an embodiment, the body of the container and film have substantially the same cross-sectional shape and color.

In an embodiment, the edible thin film has at least two colors.

In an embodiment, the edible thin film includes sides that are not parallel.

In an embodiment, the edible thin film includes a surface that is textured.

In an embodiment, at least one surface of the edible thin film includes a design.

In an embodiment, the edible film comprises fruit flavors and cooling agents to give a consumer the perception of longer lasting flavor.

In an embodiment, the edible film releases color to dye a tongue in the mouth of the consumer.

In an embodiment, the edible thin film product is so constructed and arranged as to create a tongue tattoo when dissolved in the mouth of a consumer.

In an embodiment, the edible thin film product includes ingredients that create an oral sensation as the product dissolves in the mouth of the consumer.

In an embodiment, the edible film product dissolves and breaks into smaller particles of mucoadhesive microbeads that adhere to mucosal surfaces for longer lasting breath freshening.

In an embodiment, the edible thin film product comprises a film former chosen from the group consisting of carboxymethylcellulose, methylcellulose, hydroxypropylmethylcellulose, guar gum, locust bean gum, xanthan gum, hydrolyzed gums, carrageenan, tamarind, agar agan, konjac, arabinogalactan, larch arabinogalactan, beta-glucan, algins, propylene glycol, levan, elsinan, pullulan, gellan, curdlan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

In an embodiment, the edible thin film product comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, dicalcium phosphate, and tri-calcium phosphate.

In an embodiment, the edible thin film product comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, MCTs such as neobee oil and fatty acids including palmitic, oleic, and linoleic acids.

In an embodiment, the edible thin film product includes a medicament chosen from the group consisting of pH control agents, tartar control, caries control, whitening agents, enzymes, breath freshening agents, anti-plaque/anti-gingivitis agents, saliva stimulating agents, energizing agents, pharmaceutical agents, nutraceutical agents, vitamins, mineral, other like medicaments or combinations thereof.

In an embodiment, the edible thin film product includes a flavor chosen from the group consisting of essential oils, synthetic flavors or mixtures including, but not limited to, oils derived from plants and fruits such as citrus oil, fruit essences, peppermint oil, spearmint oil, other mint oils, clove oils, oil of wintergreen, anise and the like, flavor oils with germ killing properties such as menthol, eucalyptol, thymol, like flavoring agents or combinations thereof.

In an embodiment, the edible thin film product includes an emulsifier chosen from the group consisting of lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, sorbitan tristerate, other like emulsifiers or combinations thereof.

In another embodiment of the present invention, an edible thin film comprising a film former and a flavor is provided. The edible thin film having a first portion and a second portion, the first and second portion having different colors that extend for an entire length, width, or depth of the film. In an embodiment, the edible thin film provides at least two flavors to a consumer consuming the edible thin film.

In yet a further embodiment of the present invention, an edible thin film is provided comprising a body having a shape that is indicative of a flavor of the film. In an embodiment, the film provides a consumer consuming the film with a citrus flavor or a fruit flavor.

In another embodiment of the present invention, an edible thin film is provided comprising a body having a color that is indicative of a flavor of the film. In an embodiment, the body has a color chosen from the group consisting of green, red, yellow, orange, and blue and the flavor is chosen from the group consisting of lemon, cherry, orange, lime, and grape. In an embodiment, the body has at least two colors.

In a still further embodiment of the present invention, an edible thin film is provided comprising a body that includes a surface characteristic chosen from the group consisting of a texture and a design. In an embodiment, the edible thin film includes at least two regions having a different color.

In a further embodiment of the present invention, an edible thin film is provided comprising a body that provides a consumer consuming same with an oral sensation chosen from the group consisting of temperature and gas release.

In an embodiment of the present invention, an edible thin film is provided that breaks apart and releases glitter that has a mucoadhesive coating of carboxymethylcellulose (CMC) that adheres to the tongue of the consumer for a sparkling effect.

Moreover, in an embodiment of the present invention, an edible thin film is provided that comprises at least two layers is provided.

Yet another embodiment of the present invention provides an edible thin film comprising a medicament and including on a surface thereof a design indicating at least one of the medicament, supplier, or manufacturer of the film. In an embodiment, the medicament is chosen from the group consisting of oral cleansing agents, breath freshening agents, pharmaceutical agents, nutritional agents and vitamins.

Furthermore, in an embodiment of the present invention, a method for flavoring water is provided comprising the step of providing an edible thin film having a flavor and placing the flavor in water prior to consuming the water. In an embodiment, the edible thin film has a citrus flavor.

And, in an embodiment of the present invention, a method for treating a disorder of the mouth is provided comprising the steps of providing an edible thin film including a medicament and a bioadhesive and adhering the edible thin film to a portion of the mouth that requires treatment.

A variety of methods of forming a thin edible film product can be utilized. For example, the method can include the following steps. Initially, all film-forming materials are added together with water and are agitated until all powders are mostly hydrated and few lumps are present. To this mixture, plasticizers, softening agents, colors, sweeteners, cooling agents, flavors and active ingredients are blended together to form a homogeneous solution. This solution is then fed into a hopper, and spread onto a moving substrate through a drying tunnel.

In an embodiment, the film-forming agent is a water-soluble non-starch polysaccharide.

In an embodiment, the film-forming agents include a polysaccharide and a softener.

In an embodiment, the polysaccharide is pullulan.

In another embodiment of the present invention, the film product is a vehicle for delivering active agents to a consumer.

In a further embodiment of the present invention, a method for preparing an edible film is provided comprising the steps of forming a mixture of at least one film-forming material in powder form and water, agitating the mixture until the powder is mostly hydrated and few lumps remain, adding to the mixture at least one ingredient selected from plasticizers, softening agents, colors, sweeteners, cooling agents, flavors and active ingredients, blending the mixture to obtain a homogeneous solution, spreading the solution onto a moving substrate, drying the solution on the substrate to create a flexible film, cutting the film to desired shape, and packaging the resultant product.

It is an advantage of the present invention to provide improved edible thin film products.

Furthermore, an advantage of the present invention is to provide an improved method for manufacturing edible thin film products.

Another advantage of the present invention is to provide novel forms of edible thin film product.

Still further, an advantage of the invention is that it provides an improved product/packaging design.

Moreover, an advantage of the present invention is to provide a variety of edible thin film products to enhance the marketability of same.

Further, an advantage of the present invention is to provide edible thin film products that can be used in food products and beverages.

Additionally, an advantage of the present invention is that it provides edible thin film products that can be used in methods of treating diseases or disorders.

Still further, an advantage of the present invention is to provide an improved edible thin film product including container for housing the film.

Additional features and advantages of the present invention are described in, and will be apparent from, the detailed description of the presently preferred embodiments and the drawings.

### BRIEF DESCRIPTION OF THE DRAWINGS

FIG. 1 illustrates an embodiment of the edible thin film of the present invention.

FIG. 2 illustrates another embodiment of the edible thin film of the present invention.

FIG. 3 illustrates a further embodiment of the edible thin film of the present invention.

FIG. 4 illustrates a still further embodiment of the edible thin film of the present invention.

FIG. 5 illustrates another embodiment of the edible thin film of the present invention.

FIG. 6 illustrates an embodiment of the edible thin film product in a package.

### DETAILED DESCRIPTION OF THE INVENTION

The present invention provides improved edible thin film formulations and products as well as methods of using and making same. As used herein the term “edible thin film” refers to compositions that include a film-former substrate and are designed to adhere to at least a portion of the oral cavity of a consumer and rapidly dissolve therein. “Rapidly dissolve” means that the substrate dissolves in less than 25 seconds, preferably less than 15 seconds and most preferable less than 10 seconds. To “dissolve” means to substantially lose the shape and form of the substrate. An example of an edible thin film product is the Listerine® PocketPaks™ oral care strips distributed by Pfizer.

As noted above, pursuant to the present invention edible films are provided. Edible thin films can be formed by a variety of different processes. One such process is as follows: (1) an aqueous solution is formed by blending film-forming materials together with water and are agitated until the powdered materials are mostly hydrated and few lumps are present; (2) to this mixture, plasticizers, softening agents, colors, sweeteners, cooling agents, and active ingredients are blended together to form a homogeneous solution; and (3) this solution is then applied onto a suitable carrier, and dried to form a film.

The carrier material should be impermeable to the film coating, allowing the film coating to disperse evenly onto the carrier. This also allows for ease of removal of the film from the carrier. Examples of suitable carriers include plastic or polyester films, polypropylene, polycarbonate, non-siliconized polyethylene terephthalate film, non-siliconized Kraft paper, polyethylene impregnated Kraft paper, metal belts, voltage or corona treated belts, drum dryers, and polytetrafluroethylene-impregnated glass fabric. Multiple carriers may be employed to create a multi-layered film product.

It has been found that a particularly preferred method of casting the film on the carrier may be through use of a slot die extrusion. By use of multiple extruders and specially constructed dies, it is possible to add multiple color stripes or designs to the product. Another means of creating multiple colors or designs in film product is to add dyes to the coating syrup and pass them through a rotary funnel. It is also possible to oscillate the die head to produce wavy lines on the product. The resulting films can be laminated to produce various visual effects.

The casting or applying of the solution onto a suitable carrier material can be performed using any conventional coating technique. Examples of coating techniques include spraying, dipping, comma coaters, powder coating, knife over plate, roll over roll, reverse roll, slot die extrusion, and various extrusion techniques. Pumping or air pressure may be used to feed slot dies or other extrusion processes. Film thickness can be controlled by adjusting the gap on the coating head, or by applying the desired amount of the solution onto the substrate/carrier. Also, the slot die may be serrated to produce curves or ridges on the product, giving it a unique appearance and texture.

It should be noted that no particular limitation is placed on the thickness of the film layer except that the resultant film must rapidly dissolve in the mouth of the consumer. Therefore the thickness of the film can be varied based on, for example, the desired speed of dissolution of the edible film while in the oral cavity. In particular, a thick film comprised of either pullulan or a starch derivative will dissolve slowly, without gumminess. Not only can the thickness be varied but a multi-layered film product may be provided.

After the coating step, in an embodiment, the film passes through a dryer for moisture reduction. In the dryer, drying is carried out through a variety of different means, such as high velocity turbulent hot air, conduction from steam heated slide bed, direct heating or casting of film onto a heated drum or belt, hot or cold air impingement, infrared heating, or any other suitable drying equipment that does not adversely affect the components of the film.

While in the drying system, various treatments can be applied to the solution to create a novel edible film product. For example, the solution may be exposed to a brush, giving the film texture; the solution may be separated by an air knife, thereby creating wells or creases that may aid in making a “peel and pull” type of product; the wells created by the air knife may be filled with a different type of confectionary; the solution may be sprinkled with an ingredient to add texture, flavor, or any other type of oral sensation. Further, a processing aid may be employed to reduce sticking or increase the shelf life of the product.

Once the film exits the drying system, the dried film can be either taken-up along with its substrate or peeled from the carrier to form a wide roll. As the film exits the drying system, it can be exposed to a number of different types of treatments. The film may be sprinkled with sugar, starch, flavor, color enhancers such as glitter, acids, bioadhesives, actives and texturizers such as candy sprinkles to make specialty edible thin film products directed to younger consumers.

As noted below, a wide variety of edible film products can be provided. The edible thin film can be a rolled edible thin film. Rolled edible thin films and methods of making same are disclosed in U.S. Ser. No. 10/228,742, entitled “Rolled Edible Thin Film Products and Methods of Making Same,” filed on Aug. 27, 2002, the disclosure of which is incorporated herein by reference.

On the other hand, the edible thin film can be designed to be dispensed or accessed from the container in individual servings, such as Listerine® PocketPaks™. A method for making such a product from the roll comprises creasing or perforating the film along lines generally perpendicular to the leading edge of the sheet upon exiting the drying system. Once the wide roll has been dried, and the corner has been encased along with the film layer, the wide roll is broken down into more narrow rolls according to the score lines, and may be perforated for use by the consumer.

Of course, other methods of making edible thin films can be utilized.

The present invention provides, in an embodiment, specialty edible thin films. These specialty edible thin films can be produced at various points during the film making process. An advantage of the present invention is that it can provide concepts which appeal to younger consumers. Types of specialty edible thin film products include, but are not limited to, films that are multi-flavored, multi-layered, multicolored, multi-shapes or forms, texturized, laminated, printed, graphical designs, produce “tongue-tattoos”, provide an oral sensation, have varying dissolution profiles, include bioadhesive components and combinations thereof.

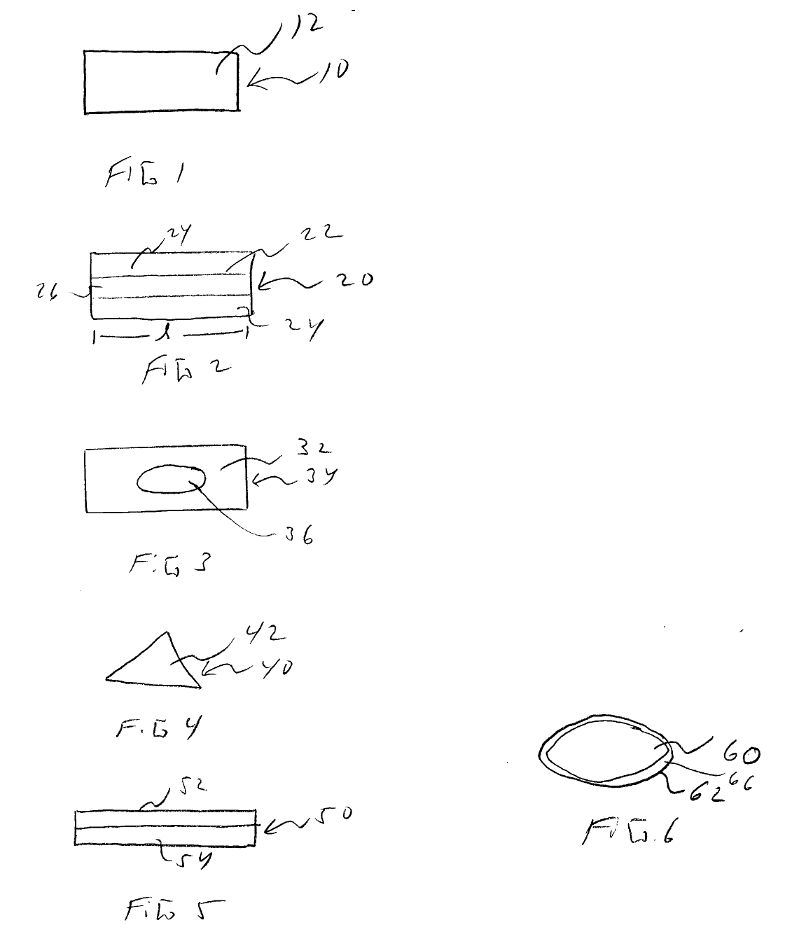

Referring now to the figures, FIG. 1 illustrates, generally, an edible film 10 of the present invention. As illustrated, the film 10 includes a body 12. As set forth in detail below, the body 12 can be constructed from a variety of components. The body 12 is designed to be placed in the mouth, specifically on the tongue of the user where it generally adheres thereto and dissolves. As noted above, a variety of edible film products are possible.

FIG. 2 illustrates an embodiment of the edible film 20. As illustrated, the body 22 comprises at least two distinct regions 24 and 26 that have a different color. Although, as illustrated in the preferred embodiment of FIG. 2, the body 22 is striped, the distinct regions run for a length “1” of the film 20, the distinct regions can take a variety of forms. For example, the distinct regions can extend for the width of the film or only the depth of the film, e.g., instead of being striped, the body 22 can have a checkerboard configuration. Likewise, it should be noted that the regions 24 and 26 do not have to be symmetrical or the same size.

It should also be noted that the product of FIG. 2 can be constructed so that the distinct regions 24 and 26, provide different flavors to the consumer or the same flavor. For example, region 24 can be yellow and provide a lemon flavor and region 26 can be red and provide a cherry flavor. If desired, the distinct regions 24 and 26 may have the same color but provide different flavors. For example, one region 24 can provide a spearmint flavor while the other region 26 provides a cinnamon flavor. Also, the distinct regions 24 and 26 may have the same or different colors, but have different formulations. For example, region 24 may be made from konjac and region 26 may be made from a modified cellulose.

Furthermore, in an embodiment, the distinct regions 24 and 26 can provide different oral sensations. For example, these sensations can comprise temperature (e.g., heat and cooling), gas release (e.g., a fizzing sensation), moisture release, or tingling or mild astringency to give a clean mouth feeling. If desired, the entire thin film can provide the oral sensation or only portions thereof. Further, it is possible for the thin film to have distinct regions that provide different sensations depending on the region. Thus, one region 24 of the film 20 may provide a heating sensation while the other region 26 provides a cooling sensation.

In a still further embodiment illustrated in FIG. 3, a surface 32 of the edible film 34 includes a design 36. This design 36 can be any desired design. For example, the design 36 can be indicative of the flavor of the edible film 34, e.g., a lemon for a lemon flavored film. Or the design 36 may be a product or company indicator, e.g., the logo of the manufacturer. The design 36 can be fanciful, seasonal, or regional, e.g., a local sports team logo. Or the design can depict a location, individual or cartoon character. Any design 36 that can be placed on the surface 32 of the film 36 using a food grade ink, color, dye, or other ingestible media can be provided.

In an embodiment, the design 36 can be transferable to the tongue of the consumer. This can be used to create a tongue tattoo. In order to create tongue tattoos, food-safe colors or dyes should be utilized to produce the design 36 on the film 34.

Referring now to FIG. 4, another embodiment of the edible thin film 40 is illustrated. The edible thin film 40 includes a body 42 that has a design that is not rectangular as in typical edible thin films such as Listerine® PocketPaks™. The body 42 can have any of a variety of cross-sectional shapes including, by way of example and not limitation, triangular, oval, circular, and hexagonal shapes. On the other hand, the body 42 can have a cross-sectional shape that is indicative of the flavor of the edible thin film 40, e.g., a lemon shape for a lemon flavored film 40. Of course, the shape of the body 42 can be fanciful if desired or representative of any other object, person, character or any other design desired.

Referring now to FIG. 5, a multilayer edible thin film 50 is illustrated. To this end, in the illustrated embodiment, the film 50 includes a top layer 52 and bottom layer 54. Although two layers 52 and 54 are illustrated, the edible thin film 50 can include more than two layers. The layers 52 and 54 can be laminated or co-extruded onto each other. The layers can provide different properties to the film 50, e.g., flavor, oral sensation, differing dissolution rates, etc.

Referring now to FIG. 6, illustrated therein is an edible thin film 60 in a container 62. The container 62 is designed to house the edible thin film so that it can be sold to consumers and dispensed therefrom. To this end, the body 64 of the container 62 defines an interior 66 for receiving and storing the film 60.

The edible thin film 60 and container 62 are designed so that the edible thin film 60 and container 62 have a shape and/or color indicative of the flavor of the film. For example, a lemon flavored film 60 can be in the shape of a lemon and the body 64 of the container 62 in the shape of a lemon. On the other hand, a lemon flavored edible thin film 60 can merely have a color indicative of its flavor, i.e., yellow. The container 62 also can have portions thereof, or the entire container, of the same color, e.g., yellow. In such an embodiment, the container 62 and/or edible thin film 60 will suggest the flavor of the product due to one of its characteristics, e.g., color or shape.

Although FIG. 6 illustrates a specific edible thin film/package structure, a variety of packaging structures can be used to house film of the present invention.

All of the above edible thin film concepts can either be used alone or in combination. Thus, a wide variety of edible thin films and products are possible.

The edible thin films of the present invention are also suitable for preparing products for food applications beyond direct consumption. For instance, they may be used to add flavors or other ingredients to soups, beverages and other consumer and commercially-prepared comestibles. For example, citrus flavor films can be provided that are added to water by a consumer to provide flavored water. A wide variety of flavors can be envisioned as well as food and beverages to which the flavored films can be added.

The edible thin film products can comprise a large number of suitable formulations. Any suitable water-soluble, film-former can be used to produce a rolled edible thin film product. Suitable film-formers include but are not limited to watersoluble non-starch polysaccharides such as carboxymethylcellulose (CMC), methylcellulose, hydroxypropylmethylcellulose (HPMC), guar gum, locust bean gum, xanthan gum, hydrolyzed gums, carrageenan, tamarind, agar agan, konjac, arabinogalactan, larch arabinogalactan, beta-glucan,-algins, propylene glycol, levan, elsinan, pullulan, pectins, curdlon, chitosan, and gum arabic; native starches such as corn starch, waxy maize starch, high-amylose corn starch, potato, tapioca, rice and wheat starch; modified starches such as those that have been acid modified, bleached, oxidized, esterified, etherified, crosslinked, and treated enzymatically; starch hydrolyzed products such as maltodextrin; protein such as albumen, gelatin, casein, salts of casein, whey, wheat gluten, zein, and protein derived from soybeans; polymers such as polyvinyl pyrrolidone, methycrylate copolymer, and carboxyvinyl copolymers alone or in any combination. In an embodiment, the concentration of the film-forming agent constitutes between 5% to about 60% by dry weight, or 20% to about 40% by dry weight of the final film composition.

Further, any suitable food-grade bulk filler can also be added to produce the film. Such fillers can reduce any slimy texture as well as provide structure to the film making it more palatable. In an embodiment, the filler can comprise approximately 1% to about 30% by dry weight of the film, or approximately 5% to about 15% by dry weight of the film. The filler can include microcrystalline cellulose, cellulose polymers, such as wood, magnesium and calcium carbonate, ground limestone, silicates, such as magnesium and aluminum silicate, clay, talc, titanium dioxide, monocalcium phosphate, di-calcium phosphate, tri-calcium phosphate, other like bulk fillers or combinations thereof.

If it is desired to use lower levels of film forming agents, softeners can also be employed to ensure the flexibility of the film, thereby reducing brittleness, and enhance ease of manufacture. The softeners, which are also known as plasticizers, may include tallow, hydrogenated tallow, hydrogenated and partially hydrogenated vegetable oils, cocoa butter, sorbitol and other polyols, glycerin, palatinit, polyethylene glycol, propylene glycol, invert sugars, corn syrup, lecithin, enzyme-modified lecithin, hydrogenated lecithin, mono-, di- and triglycerides, acetylated monoglycerides, MCTs such as neobee oil, fatty acids (e.g. stearic, palmitic, oleic, and linoleic acids), and combinations thereof. In an embodiment, the softener can constitute 0% to about 20% by dry weight of the film, or approximately 2% to about 10% by dry weight of the film.

Another means of controlling the brittleness of the film and to ensure processing efficiency, is to maintain an adequate moisture level in the film. Preferably, moisture levels should range from approximately 1% to about 20%, or approximately 5% to about 10% of the final film product.

A variety of other suitable ingredients can be added to the edible thin film of the present invention. For example, any suitable medicament for oral cleansing, breath freshening or the like can be added to the film formulation. The medicaments can include, for example, pH control agents, such as urea and buffers; inorganic components for tartar and caries control, such as phosphates, pyrophosphates, polyphosphates, and fluorides; whitening agents, enzymes such as proteolytic and glycolydic enzymes to disrupt plaque and the pellicle, enzymes such as oxidoreductase to oxidize stains on teeth and enzymes which react with sulfur compounds in breath, (such enzymes are produced by Genencor, Enzyme Development Corporation, Valley Research and Novozymes); breath freshening agents such as food acceptable metal salts such as zinc lactate, copper or zinc gluconate; anti-plaque/anti-gingivitis agents, such as chlorohexidene, CPC, and triclosan; saliva stimulating agents including, for example, food acids such as citric, lactic, malic, succinic, ascorbic, adipic, fumaric, and tartaric acids; energizing ingredients such as caffeine or guarana extract; pharmaceutical agents, nutraceutical agents, vitamins, mineral, other like medicaments or combinations thereof. Specific vitamins and minerals which are especially suited to delivery by orally-consumed films include vitamins B12, B1, B2, B6, A, D, E, folic acid, selenium, iron, and zinc.

The medicaments can be delivered or released into the oral cavity for an effective oral treatment, such oral cleansing and/or breath freshening. In this regard, the film forming agents of the edible film composition can act to entrap the medicaments within the oral cavity thereby providing extended efficacy thereof. The film product may be multi-layered, having at least one layer with an active ingredient, and at least one layer having no active ingredients. Further, medicaments having bitter notes may have their bitterness masked through different methods, particularly via microencapsulation. If desired, the edible thin film can include a bioadhesive such as polyacrylic acid, sodium carboxymethylcellulose, polyvinylpyrollidone, or combinations thereof.

If desired, the edible thin film formulations of the present invention can also include colorants or coloring agents which can be used in any suitable amount to produce a desired color. Further, the edible thin films of the present invention may have colored stripes and/or other related designs or shapes to produce color contrasts on the edible rolled film. Additional coloring may be used to intentionally dye the tongue of the consumer. Coloring agents can include, for example, natural food colors and dyes suitable for food, drug, and cosmetic applications. The colorants are typically known as FD&C dyes and lakes. Colors may be applied to film in different ways, such as adding them directly to the coating syrup and ink jetting.

A variety of flavoring agents can also be added to the rolled edible thin films. Any suitable amount and type of artificial and/or natural flavoring agents can be used in any sensorially acceptable fashion. For example, the flavor can constitute about 0.1% to about 20% by dry weight of the film, preferably approximately 10% to about 15%. The flavoring agent can include, for example, essential oils, synthetic flavors or mixtures including but not limited to oils derived from plants and fruits such as citrus oil, fruit essences, peppermint oil, spearmint oil, other mint oils, clove oils, oil of wintergreen, anise and the like, flavor oils with germ killing properties such as menthol, eucalyptol, thymol, spices such as cocoa powder and cinnamon powder, like flavoring agents or combinations thereof.

The flavor can be enhanced and distributed evenly throughout the product by emulsification. Any suitable amount and type of natural and/or synthetic food-grade emulsifier can be used. For example, the emulsifier can include lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, sorbitan tristerate, other like emulsifiers or combinations thereof.

The flavors can be emulsified by any suitable emulsification process, such as mechanical processing, vigorous stirring, intense pressure fluctuations that occur in turbulent flow such as homogenization, sonification, colloid milling and the like. Further, the flavors may also be encapsulated or spray dried and incorporated into film coating syrup or onto the edible film product to enhance flavor properties or to add texture to the film composition.

Sweetening agents may also be used in the edible film products of the present invention. Sugar sweeteners generally include saccharide-containing components including, but not limited to, sucrose, dextrose, maltose, dextrin, invert sugar, fructose, levulose, galactose, corn syrup solids, and the like, alone or in any combination. Sugarless sweeteners include, but are not limited to, sugar alcohols such as sorbitol, mannitol, xylitol, isomalt, hydrogenated starch hydrolysates, maltitol, and the like, alone or in any combination. However, the low weight of the film products of the present invention generally render these low intensity sweeteners ineffective for purposes of sweetening although they may provide functional benefits. Any suitable amount of sweetening agent can be used.

High intensity artificial sweeteners may preferably be used, alone or in combination with the above. Preferred sweeteners include, but are not limited to, sucralose, aspartame, N-substituted APM derivatives such as neotame, salts of acesulfame, alitame, saccharin and its salts, cyclamic acid and its salts, glycyrrhizin, dihydrochalones, thaumatin, monellin, and the like, alone or in any combination. In order to provide enhanced or delayed sweetness, or to provide texture to the rolled film product, it may be desirable to encapsulate the sweetener. Such techniques as wet granulation, wax granulation, spray drying, spray chilling, fluid bed coating, coacervation, and fiber extension may be used to achieve the desired characteristics.

Combinations of sugar and/or sugarless sweeteners may be used in the film product. Additionally, a softening agent may also provide additional sweetness such as with aqueous sugar or alditol solutions.

Cooling agents may also be employed in the present invention, cooling agents include, but are not limited to, menthol, WS3, WS23, Ultracool, monomenthyl succinate, alone or in any combination. Again, these cooling agents may be encapsulated or spray dried onto the film or incorporated into the coating syrup to enhance a variety of oral sensations.

Depending on the ingredients being used to make the edible thin film product, preservatives may also be employed to ensure the safety and quality of the edible thin film. Suitable preservatives include, but are not limited to, sorbic acid, sodium benzoate, potassium sorbate, methyl p-hydroxybenzoate, methyl paraben, propyl paraben, sodium propionate, and propyl p-hydroxybenzoate alone or in any combination. In addition, suitable antioxidants can also be utilized.

It should be appreciated that any suitable type, number and arrangement of process procedures or steps (e.g. mixing, heating, drying, cooling, addition of ingredients), process parameters (e.g. temperature, pressure, pH, process times) or the like can be utilized to practice the present invention.

Further, the film body can have any variety of cross-sectional shapes including, by way of example and not limitation, triangle, oval, circular, and hexagonal shapes. The shape of the film can be fanciful if desired or representative of any other object, person, character or any other design desired.

Of course, a variety of package/container designs are possible. For example, the films may be available in a multiple layered package, wherein the primary package is used to dispense the film and is combined with a secondary package that may be used to carry, protect, and be refilled with the primary package. Also, within the primary package, a few unique or a differentiated films may be included that are oriented from the end of the stack to warn the consumer that they are running low, and to purchase a refill cartridge soon. Suitable packaging materials include, but are not limited to, plastic overwrap, metal tins, blister packs, vacuum formed plastic mold, injection molded plastic or corrugated paper board. The packaging materials chosen may assist in maintaining the integrity of the product during shelf life depending on the components used in the film formula. However, it should be noted that any container design can be used as long as it allows the consumer to access the thin film.

By way of example and not limitation, the following examples illustrate various embodiments of the edible thin film formulations of the present invention.

### EXAMPLES

| | (% Dry Weight) | | | | | |
|---|---|---|---|---|---|---|
| Ingredient | Ex. 1 | Ex. 2 | Ex. 3 | Ex. 4 | Ex. 5 | Ex. 6 |
| Hydroxy- | — | 34.85 | — | 20.0 | — | — |
| propylated | | | | | | |
| Starch | | | | | | |
| Maltodextrin | — | — | 26.0 | 17.0 | — | 18.0 |
| Pullulan | 28.0 | — | — | 16.0 | 50.0 | — |
| Microcrystal- | 5.55 | 10.0 | 7.5 | — | 4.5 | 15.0 |
| line Cellulose | | | | | | |
| Carrageenan | 14.0 | 10.0 | 8.0 | 5.0 | — | 16.0 |
| Glycerin | — | 10.0 | — | 3.0 | 9.5 | 6.0 |
| Propylene | 9.0 | — | 6.0 | 3.0 | — | — |
| Glycol | | | | | | |
| Sodium | 14.0 | 8.0 | 23.0 | 7.0 | — | 25.5 |
| Alginate | | | | | | |
| Lecithin | — | — | 1.0 | — | — | — |
| Sucralose | 1.0 | 2.0 | 0.50 | — | 0.50 | — |
| Neotame | — | — | — | — | 1.0 | 0.45 |
| Aspartame | 0.45 | — | 1.5 | 1.0 | 0.50 | 1.0 |
| Wintergreen | — | — | — | 10.0 | — | — |
| Flavor | | | | | | |
| Spearmint | — | — | 14.0 | — | — | 10.0 |
| Menthol | — | — | — | 4.9 | 6.3 | 2.0 |
| Cherry Flavor | 12.0 | — | — | — | 8.0 | — |
| Lemon Flavor | 2.0 | 15.0 | 2.0 | — | 4.5 | — |
| Citric Acid | 1.0 | — | — | — | 0.50 | — |
| WS-3 | 2.0 | — | 0.50 | 1.0 | 1.5 | — |
| Color | 0.05 | 0.15 | 0.25 | 0.10 | 0.20 | 0.05 |
| Water | 10.95 | 10.0 | 9.75 | 12.0 | 13.0 | 6.0 |
| TOTAL | 100.0 | 100.0 | 100.0 | 100.0 | 100.0 | 100.0 |
| | (% Dry Weight) | | | | | |
| Ingredient | Ex. 7 | Ex. 8 | Ex. 9 | Ex. 10 | Ex. 11 | Ex. 12 |
| Maltodextrin | 23.0 | — | — | — | 30.0 | — |
| PURE- | — | — | 46.0 | — | — | — |
| COTE ® | | | | | | |
| B790* | | | | | | |
| Acid Thinned | — | — | — | 32.0 | — | 20.0 |
| Corn Starch | | | | | | |
| Levan | — | 21.0 | — | 10.0 | — | — |
| Alginate | 26.0 | 17.0 | — | 5.0 | — | 3.5 |
| Carrageenan | 5.0 | 14.0 | 11.0 | — | 6.0 | 2.5 |
| Gelatin | — | 5.0 | — | — | — | 23.0 |
| Polyvinyl | — | — | — | 8.0 | — | 15.0 |
| Pyrollidone | | | | | | |
| Microcrystal- | 8.0 | 3.0 | — | — | 6.0 | 1.2 |
| line Cellulose | | | | | | |
| Silica | — | — | 5.0 | — | — | — |
| Calcium | — | — | — | — | 10.25 | 5.3 |
| Phosphate | | | | | | |
| Glycerin | — | — | — | 6.5 | 2.0 | 1.5 |
| Locust Bean | — | — | 7.5 | — | — | 3.0 |
| Gum | | | | | | |
| Soybean Oil | 16.0 | — | — | 2.0 | 6.0 | — |
| Vegetable Oil | — | 10.0 | — | — | 6.0 | — |
| Lecithin | 0.50 | — | 1.0 | 2.0 | — | 0.20 |
| Sorbitol | — | — | 4.5 | — | — | 2.0 |
| Corn Syrup | — | — | | 4.5 | — | — |
| Dextrose | — | — | — | 8.0 | — | 1.0 |
| Neotame | — | — | — | 1.0 | — | 0.25 |
| Saccharin | — | 3.0 | — | — | — | — |
| Sucralose | — | — | 1.50 | — | 3.0 | — |
| Menthol | 5.0 | — | 3.0 | 1.0 | — | — |
| Peppermint | 8.0 | — | — | — | — | — |
| Cinnamon | — | — | — | 10.0 | — | — |
| Anise | — | — | 9.0 | — | — | — |
| Eucalyptol | 1.0 | 3.0 | 0.50 | — | — | — |
| Methyl | — | 12.75 | 1.0 | — | — | — |
| Salicylate | | | | | | |
| Grape Flavor | — | — | — | — | 13.0 | — |
| Melon Flavor | — | — | — | — | — | 11.0 |
| WS-23 | — | 2.0 | — | 0.50 | — | — |
| Ultracool | — | — | 0.50 | 0.50 | — | — |
| Adipic Acid | — | — | — | — | 2.50 | — |
| Color | 0.30 | 0.25 | 0.50 | — | 0.35 | 0.55 |
| Water | 7.20 | 10.0 | 8.50 | 9.0 | 14.9 | 10.0 |
| TOTAL | 100.0 | 100.0 | 100.0 | 100.0 | 100.0 | 100.0 |

*PURE-COTE ® B790 is a product of the Grain Processing Corporation
(GPC), located in Muscatine, Iowa. PURE-COTE ® B790 is a
flash-dried modified corn starch designed specifically for applications
with very low viscosity.

| | (% Dry Weight) | | | | | |
|---|---|---|---|---|---|---|
| Ingredient | Ex. 13 | Ex. 14 | Ex. 15 | Ex. 16 | Ex. 17 | Ex. 18 |
| Hydroxy- | 27.0 | 15.0 | — | — | — | — |
| propylmethyl- | | | | | | |
| cellulose | | | | | | |
| (HPMC) | | | | | | |
| PURE- | — | — | — | 48.0 | 21.0 | — |
| COTE ® | | | | | | |
| B792* | | | | | | |
| Gum Arabic | — | 25.0 | 5.0 | — | — | — |
| Pectin | 12.0 | — | 23.0 | — | — | — |
| Chitosan | — | 5.0 | — | — | 30.0 | — |
| Oxidized | — | — | 5.5 | — | — | 25.0 |
| Potato Starch | | | | | | |
| Waxy Maize | — | 11.5 | 2.5 | — | — | — |
| Starch | | | | | | |
| Alginate | — | — | — | 22.0 | 6.0 | — |
| Casein | — | — | 3.0 | — | — | 25.0 |
| Wheat Gluten | — | — | 8.0 | — | 2.0 | — |
| Carboxyvinyl | 15.0 | — | — | — | — | — |
| Copolymer | | | | | | |
| Aluminum | — | 7.0 | — | — | 12.0 | — |
| Silicate | | | | | | |
| Calcium | 5.0 | — | 15.0 | — | — | 4.5 |
| Carbonate | | | | | | |
| Titanium | — | 2.50 | — | — | — | 4.5 |
| Dioxide | | | | | | |
| Hydrogenated | — | — | — | 2.0 | — | — |
| Lecithin | | | | | | |
| Cocoa Butter | 15.0 | — | — | — | 3.5 | 1.5 |
| Linoleic Acid | — | 2.0 | 5.5 | — | 4.5 | — |
| Orange Mint | 15.0 | — | | — | 4.0 | — |
| Flavor | | | | | | |
| Wild Berry | — | 10.50 | — | — | 5.0 | — |
| Flavor | | | | | | |
| Strawberry | — | — | — | — | — | 12.25 |
| Flavor | | | | | | |
| Clove Oil | — | — | 2.0 | — | — | — |
| Pepper | — | — | 0.40 | — | — | — |
| Peppermint | — | — | — | 9.15 | — | — |
| Eucalyptol | — | — | — | 1.0 | — | — |
| Cardamom | — | — | 8.0 | — | — | — |
| Sorbitan | 1.0 | — | — | — | — | — |
| Monopalmitate | | | | | | |
| Citric Acid | — | — | — | — | — | 6.0 |
| Xylitol | 2.0 | — | 1.0 | — | — | — |
| Malitol | — | — | 3.25 | — | 0.5 | — |
| Fructose | — | — | 2.0 | — | — | 5.0 |
| Glycyrrhizin | — | 1.0 | — | — | 0.5 | — |
| Acesulfame K | — | 1.25 | 0.25 | — | — | — |
| Encapsulated | 0.50 | — | — | 1.25 | — | — |
| Aspartame | | | | | | |
| Liquid Sorbitol | 1.0 | 3.25 | — | 7.0 | — | 2.0 |
| Monomethyl | — | 0.50 | — | — | — | 1.0 |
| Succinate | | | | | | |
| BHT | — | 1.0 | — | — | — | 0.25 |
| Color | 0.50 | 1.0 | 0.50 | 0.35 | 0.25 | 0.15 |
| Water | 6.0 | 13.5 | 15.1 | 9.25 | 10.75 | 12.85 |
| TOTAL | 100.0 | 100.0 | 100.0 | 100.0 | 100.0 | 100.0 |

*PURE-COTE ® B792 is a product of the Grain Processing Corporation
(GPC), located in Muscatine, Iowa. PURE-COTE ® B792 is a
pregelatinized modified corn starch designed specifically for applications
requiring low viscosity.

| | (% Dry Weight) | | | | | |
|---|---|---|---|---|---|---|
| Ingredient | Ex. 19 | Ex. 20 | Ex. 21 | Ex. 22 | Ex. 23 | Ex. 24 |
| Maltodextrin | 45.0 | — | — | 22.0 | — | 18.0 |
| Pullulan | — | 26.0 | — | — | — | 15.0 |
| Alginate | 2.0 | — | 29.5 | — | — | — |
| Carrageenan | — | — | 18.0 | — | 55.0 | — |
| High Amylose | 10.0 | 6.5 | — | 8.0 | — | — |
| Corn Starch | | | | | | |
| Whey Protein | — | — | 9.0 | — | — | 3.0 |
| Zein | — | 19.0 | — | 20.0 | — | 27.5 |
| Methylcrylate | — | 14.0 | — | 10.0 | — | 1.5 |
| Copolymer | | | | | | |
| Ethyl Cellulose | 7.0 | — | — | — | 17.0 | — |
| Magnesium | 4.0 | — | — | — | — | — |
| Silicate | | | | | | |
| Tri-Calcium | 4.0 | — | — | — | — | — |
| Phosphate | | | | | | |
| Hydrogenated | 3.0 | — | — | — | — | — |
| Tallow | | | | | | |
| Sucralose | — | — | — | 1.5 | 1.0 | 3.0 |
| Encapsulated | — | — | — | — | 1.0 | — |
| Aspartame | | | | | | |
| Neotame | 2.5 | 3.0 | 1.0 | — | — | — |
| Sorbitol | 1.50 | — | — | 1.0 | — | — |
| Maltose | — | 4.5 | — | 2.5 | — | — |
| WS-3 | — | 2.9 | — | 1.0 | — | 4.0 |
| Menthol | 8.0 | 2.0 | — | 2.0 | — | — |
| Rose Oil | — | 2.0 | — | — | — | — |
| Honey Lemon | — | — | — | 6.0 | — | — |
| Flavor | | | | | | |
| Tangerine | — | — | — | 5.0 | — | 15.0 |
| Flavor | | | | | | |
| Lime Flavor | — | — | — | — | 12.0 | — |
| Methyl | — | — | 10.0 | — | — | — |
| Salicylate | | | | | | |
| Eucalyptol | 2.0 | 7.5 | 4.95 | — | — | — |
| Tea Tree Oil | — | — | 10.0 | — | — | — |
| Grapeseed | — | 3.0 | — | — | — | — |
| Extract | | | | | | |
| Vitamin C | — | — | — | 2.5 | — | — |
| Vitamin A, B, | — | — | 3.5 | — | — | — |
| D, E Complex | | | | | | |
| Zinc Gluconate | — | — | — | 5.5 | — | — |
| Sodium | — | — | — | — | 0.15 | — |
| Selenite | | | | | | |
| Encapsulated | — | — | — | — | 2.0 | — |
| Reduced Iron | | | | | | |
| Malic Acid | — | — | — | 3.5 | — | 0.50 |
| Echinacea | — | — | 1.0 | — | — | — |
| Cetyl | 3.0 | — | — | — | — | — |
| Pyridinium | | | | | | |
| Chloride | | | | | | |
| Encapsulated | — | 3.0 | — | — | — | — |
| Caffeine | | | | | | |
| Sodium | — | — | 0.5 | — | — | — |
| Benzoate | | | | | | |
| BHA | — | — | 1.0 | — | — | 0.30 |
| Color | — | 0.1 | 0.55 | 0.45 | 0.30 | 0.20 |
| Water | 8.0 | 6.5 | 11.0 | 9.05 | 11.55 | 7.0 |
| TOTAL | 100.0 | 100.0 | 100.0 | 100.0 | 100.0 | 100.0 |

In the above examples as well as the examples set forth below, the method of making the edible thin film is generally as set forth in the section entitled preparation method set forth infra.

### Example 25

Upon exiting the drying system, powdered citric acid is dispersed evenly upon the dried film layer of Example 14.

### Example 26

Flavor crystals are sprinkled onto the coating solution while in the drying chamber of Example 4.

### Example 27

While exiting the drying system, the dried film layer of Example 6 is stamped, leaving the imprint of a company logo.

### Example 28

1.5% proteolytic enzyme is added to the film formula of Example 1.

### Example 29

Examples 1, 2, and 12 are employed to make a multi-flavored, multi-layered and multi-colored edible film product.

### Example 30

Examples 7, 8, and 19 are employed to make a multi-flavored and multi-layered edible film product that offers a dental benefit.

### Example 31

Example 30 is employed, wherein one film layer has a thickness of 10 microns, one film layer has a thickness of 30 microns, and one film layer has a thickness of 60 microns.

### Example 32

The coating solution of Example 11 is released through multiple side by side die heads in which each die head contains the same coating formulation but has different coloring added. As the different colored solutions are released onto a moving substrate, a comb-like separator inhibits the mixing of the solutions to yield a striped edible film product.

### Example 33

Using example 32, the solution is dried to the moving substrate and the dried film is packaged with the substrate to create a “peel and pull” edible film product.

### Example 34

Using Example 2, upon exiting the drying system, die cutting is employed to make a lemon-shaped yellow edible film product.

### Example 35

Using Example 20, while moving through the drying system, die cutting is employed to make a coffee-cup shaped toffee-colored edible film product.

### Example 36

As the coating solution of Example 13 is released onto the substrate, the roller employed has ridges on it to create a textured, rippled effect on the edible film product.

### Example 37

Using Example 34, the product is packaged in a container having a body that is lemon shaped.

### Example 38

Using Example 31, one film layer is modified so that it substantially adheres to the oral mucosa of the consumer.

### Example 39

The edible film product of Example 19 is modified by removing a high amylose corn starch and adding polyacrylic acid so that it substantially adheres to the tooth of a consumer.

### Example 40

Using Example 23, the film is marketed so that a consumer is encouraged to dissolve the film in a beverage to add flavor to a beverage.

Preparation Method:

1) Blending:

a) Powdered materials (such as film-forming agents) are blended together using a ribbon blender or equivalent.

b) Flavors and flavor components/enhancers are blended together using mechanical agitation or equivalent.

2) Mixing:

a) Add powdered mix to a mixing tank filled with the appropriate amount of water.

b) After the powdered mix is mostly hydrated, increase the temperature and add softening agents, color, and sweetener in succession while the solution temperature is raising. Maintain the blend at an even temperature, about 105-115° F.

c) The conditions of the mixing room are about 70-80° F., 40-50%RH.

3) Drying:

a) Feed the solution into a feed hopper.

b) Upon entering the drying system, the film is perforated along lines generally perpendicular to the leading edge of the sheet using a comb or bar.

c) Adjust heater temperature to achieve an exit film temperature of about 215-220° F. This should produce a film having a moisture of about 9-11%.

d) Adjust coma roll to produce a dry thickness of about 48-52 microns.

e) Drying room conditions are about 70-80° F., 40-50%RH.

4) Separating:

a) The take-up roll is then broken down into narrower rolls along the score lines created while in the drying system.

b) The film roll is then unwound from the substrate and packaged as desired.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. An edible thin film product comprising:
a container including a body that defines an interior for housing the edible thin film;
the edible thin film having a characteristic indicative of a flavor of the edible thin film, the characteristic being chosen from the group consisting of color and shape; and
the body including the characteristic.

2. The edible thin film product of claim 1 wherein the body has substantially the same cross-sectional shape as the edible thin film.

3. The edible thin film product of claim I wherein the body has portions thereof that are of a same color as the edible thin film.

4. The edible thin film product of claim 1 wherein the edible thin film provides at least two flavors.

5. The edible thin film product of claim 1 wherein the body and film have substantially the same cross-sectional shape and color.

6. The edible thin film product of claim 1 wherein the edible thin film has at least two colors.

7. The edible thin film product of claim 1 wherein the edible thin film includes sides that are not parallel.

8. The edible thin film product of claim 1 wherein the edible thin film includes a surface that is textured.

9. The edible thin film product of claim 1 wherein at least one surface of the edible thin film includes a design.

10. The edible thin film product of claim 1 wherein the edible film releases color to dye a tongue in the mouth of the consumer.

11. The edible thin film product of claim I wherein the edible thin film is so constructed and arranged so as to create a tongue tattoo when dissolved in the mouth of a consumer.

12. The edible thin film product of claim I wherein the edible thin film includes ingredients that create an oral sensation as the film dissolves in the mouth of the consumer.

13. The edible thin film product of claim 1 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, pullulan, levan, elsinan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

14. The edible thin film product of claim 1 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

15. The edible thin film product of claim 1 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

16. The edible thin film product of claim 1 wherein the edible thin film includes a flavor chosen from the group consisting of essential oils, citrus oil, fruit essences, spices, peppermint oil, spearmint oil, mint oils, clove oils, oil of wintergreen, anise, flavor oils, with germ killing properties including menthol, eucalyptol, thymol, and synthetic flavors.

17. The edible thin film product of claim 1 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

18. An edible thin film comprising a film former and flavor, the edible thin film having a first portion and a second portion, the first and second portion having different colors that extend for an entire length, width, or depth of the film.

19. The edible thin film of claim 18 wherein the edible thin film provides at least two flavors to a consumer consuming the edible thin film.

20. The edible thin film of claim 18 wherein the edible thin film includes sides that are not parallel.

21. The edible thin film of claim 18 wherein the edible thin film includes a surface that is textured.

22. The edible thin film of claim 18 wherein at least one side of the edible thin film includes a design.

23. The edible thin film of claim 18 wherein the edible film releases color to dye a tongue of the consumer when dissolved in the mouth of a consumer.

24. The edible thin film of claim 18 wherein the edible thin film is so constructed and arranged so as to create a tongue tattoo when dissolved in the mouth of a consumer.

25. The edible thin film of claim 18 wherein the edible thin film includes ingredients that create an oral sensation as the film dissolves in the mouth of a consumer.

26. The edible thin film of claim 18 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, levan, elsinan, pullulan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

27. The edible thin film of claim 18 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

28. The edible thin film of claim 18 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

29. The edible thin film of claim 18 wherein the edible thin film includes a flavor chosen from the group consisting of essential oils, synthetic flavors, citrus oil, fruit essences, spices, peppermint oil, spearmint oil, mint oils, clove oils, oil of wintergreen, anise, flavor oils with germ killing properties including menthol, eucalyptol, and thymol.

30. The edible thin film of claim 18 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

31. An edible thin film comprising a body defined, at least in part, by a film-former and at least one flavor, the body including at least two separate and distinct regions that provide a different characteristic to a consumer placing the edible thin film in the consumer's mouth.

32. The edible thin film of claim 31 wherein the characteristic is a flavor.

33. The edible thin film of claim 31 wherein the characteristic is a mouth sensation.

34. The edible thin film of claim 31 wherein the mouth sensation is chosen from the group consisting of temperature sensation, gas generation, and moisture release.

35. The edible thin film of claim 31 wherein the distinct regions extend for an entire length or width of the edible thin film.

36. The edible thin film of claim 31 wherein the regions have different colors.

37. The edible thin film of claim 31 wherein the edible thin film includes sides that are not parallel.

38. The edible thin film of claim 31 wherein the regions include surfaces that have a different texture.

39. The edible thin film of claim 31 wherein at least one side of the edible thin film includes a design.

40. The edible thin film of claim 31 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, levan, elsinan, pullulan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

41. The edible thin film of claim 31 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

42. The edible thin film of claim 31 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

43. The edible thin film of claim 31 wherein the edible thin film includes a flavor chosen from the group consisting of essential oils, synthetic flavors, citrus oil, fruit essences, spices, peppermint oil, spearmint oil, mint oils, clove oils, oil of wintergreen, anise and the like, flavor oils with germ killing properties including menthol, eucalyptol, and thymol.

44. The edible thin film of claim 31 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

45. An edible thin film comprising a body having a shape that is indicative of a flavor of the film.

46. The edible thin film of claim 45 wherein the film provides a consumer consuming the film with a citrus flavor.

47. The edible thin film of claim 45 wherein the film provides a consumer consuming the film with a fruit flavor.

48. The edible thin film of claim 45 wherein the edible thin film includes a surface that is textured.

49. The edible thin film of claim 45 wherein at least one surface of the edible thin film includes a design.

50. The edible thin film of claim 45 wherein the edible film releases color to dye a tongue in the mouth of the consumer.

51. The edible thin film of claim 45 wherein the edible thin film is so constructed and arranged so as to create a tongue tattoo when dissolved in the mouth of a consumer.

52. The edible thin film of claim 45 wherein the edible thin film includes ingredients that create an oral sensation as the film dissolves in the mouth of the consumer.

53. The edible thin film of claim 45 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, pullulan, levan, elsinan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

54. The edible thin film of claim 45 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

55. The edible thin film of claim 45 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

56. The edible thin film of claim 45 wherein the edible thin film includes a flavor chosen from the group consisting of essential oils, citrus oil, fruit essences, spices, peppermint oil, spearmint oil, mint oils, clove oils, oil of wintergreen, anise, flavor oils, with germ killing properties including menthol, eucalyptol, thymol, and synthetic flavors.

57. The edible thin film of claim 45 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

58. An edible thin film comprising a body having a color that is indicative of a flavor of the film.

59. The edible thin film of claim 58 wherein the body has a color chosen from the group consisting of green, red, yellow, orange, and blue.

60. The edible thin film of claim 58 wherein the flavor is chosen from the group consisting of lemon, cherry, orange, lime, and grape.

61. The edible thin film of claim 58 wherein the body has at least two colors.

62. The edible thin film of claim 58 wherein the edible thin film includes sides that are not parallel.

63. The edible thin film of claim 58 wherein the edible thin film includes a surface that is textured.

64. The edible thin film of claim 58 wherein the edible thin film releases color to dye the tongue of the consumer.

65. The edible thin film of claim 58 wherein at least one side of the edible thin film product includes a design.

66. The edible thin film of claim 58 wherein the edible thin film is so constructed and arranged so as to create a tongue tattoo when dissolved in the mouth of a consumer.

67. The edible thin film of claim 58 wherein the edible thin film includes ingredients that create an oral sensation as the product dissolves in the mouth of the consumer.

68. The edible thin film of claim 58 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, pullulan, levan, elsinan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

69. The edible thin film of claim 58 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

70. The edible thin film of claim 58 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

71. The edible thin film of claim 58 wherein the edible thin film includes a flavor chosen from the group consisting of essential oils, citrus oil, fruit essences, spices, peppermint oil, spearmint oil, mint oils, clove oils, oil of wintergreen, anise, flavor oils, with germ killing properties including menthol, eucalyptol, thymol, and synthetic flavors.

72. The edible thin film of claim 58 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

73. An edible thin film comprising a body that includes a surface characteristic chosen from the group consisting of a texture and a design and includes at least two regions having a different color.

74. An edible thin film comprising a body that includes at least two regions having a different color and the film providing a consumer consuming the edible thin film with an oral sensation chosen from the group consisting of temperature and gas release.

75. An edible thin film comprising a medicament and including on a surface thereof a design indicating at least one of the medicament, supplier, and manufacturer of the film.

76. The edible thin film of claim 75 wherein the medicament is chosen from the group consisting of oral cleansing agents, breath freshening agents, pharmaceutical agents, nutritional agents and vitamins.

77. An edible thin film comprising a body including on a surface thereof a design.

78. The edible thin film of claim 77 wherein the edible thin film includes a surface that is textured.

79. The edible thin film of claim 77 wherein the edible film releases color to dye a tongue of the consumer when dissolved in the mouth of a consumer.

80. The edible thin film of claim 77 wherein the edible thin film is so constructed and arranged so as to create a tongue tattoo when dissolved in the mouth of a consumer.

81. The edible thin film of claim 77 wherein the edible thin film includes ingredients that create an oral sensation as the film dissolves in the mouth of a consumer.

82. The edible thin film of claim 77 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, levan, elsinan, pullulan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

83. The edible thin film of claim 77 wherein the edible thin film comprises a filler chosen from the group consisting of microcrystalline .cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

84. The edible thin film of claim 77 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

85. The edible thin film of claim 77 wherein the edible thin film includes a flavor chosen from the group consisting of essential oils, synthetic flavors, citrus oil, fruit essences, spices, peppermint oil, spearmint oil, mint oils, clove oils, oil of wintergreen, anise, flavor oils with germ killing properties including menthol, eucalyptol, and thymol.

86. The edible thin film of claim 77 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

87. An edible thin film comprising a body that is defined by at least two layers.

88. The edible thin film of claim 87 wherein the edible thin film provides at least two flavors to a consumer consuming the edible thin film.

89. The edible thin film of claim 87 wherein the edible thin film includes a surface that is textured.

90. The edible thin film of claim 87 wherein at least one side of the edible thin film includes a design.

91. The edible thin film of claim 87 wherein the edible film releases color to dye a tongue of the consumer when dissolved in the mouth of a consumer.

92. The edible thin film of claim 87 wherein the edible thin film is so constructed and arranged so as to create a tongue tattoo when dissolved in the mouth of a consumer.

93. The edible thin film of claim 87 wherein the edible thin film includes ingredients that create an oral sensation as the film dissolves in the mouth of a consumer.

94. The edible thin film of claim 87 wherein the edible thin film comprises a film former chosen from the group consisting of carboxymethylcellulose, methylcellulose, hydroxypropylmethylcellulose, guar gum, locust bean gum, xanthan gum, carrageenan, konjac, arabinogalactan, beta-glucan, algins, propylene glycol, levan, elsinan, pullulan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

95. The edible thin film of claim 87 wherein the edible thin film comprises a filler chosen from the group consisting of microcrystalline cellulose, cellulose polymers, including wood, magnesium and calcium carbonate, ground limestone, silicates, including magnesium and aluminum silicate, clay, talc, titanium dioxide, mono-calcium phosphate, di-calcium phosphate, and tri-calcium phosphate.

96. The edible thin film of claim 87 wherein the edible thin film comprises a plasticizer chosen from the group consisting of tallow, hydrogenated tallow, hydrogenated and partially hydrogenated vegetable oils, cocoa butter, sorbitol, glycerin, polyethylene glycol, propylene glycol, invert sugars, corn syrup, lecithin, hydrogenated lecithin, enzyme-modified lecithin, mono-, di- and triglycerides, acetylated monoglycerides, and fatty acids including palmitic, oleic, and linoleic acids.

97. The edible thin film of claim 87 wherein the edible thin film includes a flavor chosen from the group consisting of essential oils, synthetic flavors, citrus oil, fruit essences, spices, peppermint oil, spearmint oil, mint oils, clove oils, oil of wintergreen, anise, flavor oils with germ killing properties including menthol, eucalyptol, and thymol.

98. The edible thin film of claim 87 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

99. The edible thin film of claim 87 wherein the layers have differing rates of dissolution.

100. A method for flavoring water comprising the step of providing an edible thin film having a flavor and placing the flavor in water prior to consuming the water.

101. The method of claim 100 wherein the edible thin film has a citrus flavor.

102. The edible thin film of claim 100 wherein the edible thin film comprises a film former chosen from the group consisting of carboxymethylcellulose, methylcellulose, hydroxypropylmethylcellulose, guar gum, locust bean gum, xanthan gum, hydrolyzed gums, carrageenan, konjac, arabinogalactan, beta-glucan, algins, propylene glycol, pullulan, levan, elsinan, pectins, chitosan, gum arabic, native starches including corn starch, waxy maize starch, high-amylose corn starch, potato, tapioca, rice and wheat starch, modified starches including acid modified, bleached, oxidized, esterified, etherified, crosslinked, and enzymatically-treated starches; starch hydrolyzed products including maltodextrin; protein including albumen, gelatin, casein, salts of casein, whey, wheat gluten, zein, and protein derived from soybeans; polymers including polyvinyl pyrrolidone, methycrylate copolymer, and carboxyvinyl copolymers.

103. The edible thin film of claim 100 wherein the edible thin film includes an emulsifier chosen from the group consisting of lecithin, enzyme-modified lecithin, food-grade non-ionic emulsifiers, such as fatty acids (C₁₀-C₁₈), mono and diacyl glycerides, ox bile extract, polyglycerol esters, polyethylene sorbitan esters, propylene glycol, sorbitan monopalmitate, and sorbitan tristerate.

104. The edible thin film of claim 100 wherein at least one side of the edible thin film product includes a design.

105. A method for treating a disorder of the mouth comprising the steps of providing an edible thin film including a medicament and adhering the edible thin film to a portion of the mouth exhibiting the disorder.

106. The method of claim 105 wherein the edible thin film includes a bioadhesive.
